(19) United States
(12) Patent Application Publication
(10) Pub. No.: US 2022/0142806 A1
(43) Pub. Date: May 12, 2022

(54) Urine Collecting System Interventions For Improving Kidney Function

(71) Applicant: NXT Biomedical, LLC, Irvine, CA (US)

(72) Inventors: Joseph Passman, Irvine, CA (US); Abubaker Khalifa, Irvine, CA (US); Stanton J. Rowe, Irvine, CA (US); Glen Rabito, Irvine, CA (US); Robert C. Taft, Irvine, CA (US); Alexander Siegel, Irvine, CA (US)

(73) Assignee: NXT Biomedical, LLC, Irvine, CA (US)

(21) Appl. No.: 17/595,386

(22) PCT Filed: May 18, 2020
(86) PCT No.: PCT/US20/33460

§ 371 Date: Nov. 15, 2021

Related U.S. Application Data
(60) Provisional application No. 62/849,775, filed on May 17, 2019.

(51) Int. Cl. A61F 5/442; A61M 1/00; A61M 3/02; A61M 25/00

(52) U.S. Cl. CPC A61F 5/442; A61M 1/85; A61M 3/0254; A61M 2025/1052; A61M 25/0068; A61M 2025/0002; A61M 2025/0073; A61M 3/0279

(57) ABSTRACT

Renal and urine collection system interventions are provided that improve kidney function by manipulating pressures and/or infusing therapeutic agents in the renal pelvis of the kidney or kidneys. Setting a vacuum and/or infusing therapeutic agents in the renal pelvis results in increased glomerular filtration rate, general solute clearance, and free water excretion via the kidney through the urinary tract and is useful for treatment of CHF, ADHF, AKI, CKD, and many other conditions characterized by fluid overload by reducing fluid buildup. The fluid drawn from the renal pelvis or pelvises is delivered to an external or implanted reservoir or to the bladder using flow manipulation mechanism such as one-way valves, occlusion balloons, multi-lumen catheters, and other mechanisms. Pumps are provided for use in establishing the vacuum pressures that are either manual or motorized.

### RELATED APPLICATIONS

This application claims priority to U.S. Provisional Application Ser. No. 62/849,775 filed May 17, 2019 entitled Ureteral Interventions For Improving Kidney Function, which is incorporated by reference herein in its entirety.

### BACKGROUND OF THE INVENTION

Congestion in the kidneys can result from low cardiac output, tubuloglomerular feedback, increased intra-abdominal pressure, increased venous pressure, and other conditions. This congestion can lead to Acute Kidney Injury (AKI), which is a sudden episode of kidney failure or kidney damage that occurs rapidly, over the course of a few days or even a few hours. AKI causes a build-up of waste products in the blood and reduces the ability of the kidneys to maintain a proper fluid balance in the body, which may lead to adverse effects on the brain, heart, lungs and other organs.

AKI is very common among hospital patients, especially elderly hospital patients, and represents a massive burden on the health care system, partly due to the complex nature of present treatment therapies. For example, when AKI is a complication of systemic illness, fluid administration is often considered essential to prevent hemodynamic and nephrotoxic insults that might further compromise renal function. A long-standing tenet of AKI management has promoted volume resuscitation in response to hypotension and oliguria to augment cardiac output and urine output, respectively. The benefit of this approach is challenged, however, by increasing evidence suggesting that fluid overload is associated with impaired organ function and generally, iatrogenic morbidity and mortality. Clinicians treating AKI thus have difficulties balancing the need to give fluids to maintain blood pressure while knowing that fluid overload drives mortality and morbidity. Fluid overload in critically ill patients is an iatrogenic consequence of resuscitation. However, resuscitation is critical to treat the hypotension and the systemic inflammatory response.

The attending physician managing AKI is therefore faced with the following scenario: 1) Fluids are required to resuscitate the patient. 2) The patient's kidneys are not functional and cannot offload the administered fluid. 3) The patient is placed on dialysis, a form of renal replacement therapy (RRT). 4) Patients on dialysis have a risk of intradialytic hypotension limiting ultrafiltration and treatment success. In addition to hypotension, RRT has limitations such as waiting for the kidney to gain physiologic function, not promoting renal recovery, expense, time, etc.

There is thus a significant need for a treatment for AKI that promotes renal recovery, ultrafiltration, and/or solute clearance in AKI using the native organ. There have been disclosures that involve applying negative pressures to the urine collecting system to improve volume off-loading and solute clearance. There have also been disclosures of systems and devices for retrograde ureteral access and application of negative pressure to the renal pelvis. One example of such a disclosure is U.S. Pat. 6,500,158, entitled Method of Inducing Negative Pressure in the Urinary Collecting System and Apparatus Therefor, filed on Mar. 26, 1998 by Ikeguchi. However, these disclosures do not discuss, or make allowances for, the application of therapeutic agents in addition to the application of vacuum pressures.

Therapeutic agents could be an advantageous addition to the application of negative renal pelvic pressures. Not only do therapeutic agents provide various direct chemical treatments, infusing chemical agents into the renal pelvis may increase the production of urine and solute clearance by the nephrons due to contact with a fluid that has a different solute concentration, in an attempt to equalize the concentrations. It is also possible that these therapeutic agents limit antidiuretic hormone-mediated reabsorption of water in the urine collection system.

Other conditions that may be treated by increasing renal output include: Acute Decompensated Heart Failure, Chronic Heart Failure, Chronic Kidney Disease, Acute Kidney Disease, Syndrome of Inappropriate Antidiuretic Hormone Secretion (SIADH—also known as dilutional hyponatremia), Cerebral/Renal Salt Wasting Syndrome, Cirrhosis with refractory ascites requiring regular large volume paracentesis, and other nephrotic syndromes. Additionally, there are some nephrotic syndromes that are associated with poor renal function and therefore require acute initiation of hemodialysis for volume control.

### OBJECTS AND SUMMARY OF THE INVENTION

The present invention is directed to meeting the aforementioned needs by providing devices and method for reestablishing renal perfusion, enhancing primary filtrate formation, and augmenting total urine output and solute clearance, allowing the kidneys to return to normal function through the use of vacuum pressures and/or therapeutic agents.

More specifically, various devices and methods are disclosed herein that improve the milieu of the urine collection, glomerular, and medullary anatomy to increase volume off-loading and solute clearance. Several embodiments access the renal pelvis or general urine collection system through a retroperitoneal approach (i.e. a nephrostomy-style access). Other embodiments are delivered to the renal pelvis or general urine collection system via a retrograde ureteral access. Still other embodiments are delivered to the renal pelvis or urine collection system via the cardiovascular system using percutaneous methods.

These access routes are then used to provide therapeutic regimens to the renal pelvis, such as negative pressure and/or intermittent perfusion of diuretic agents (such as furosemide or antidiuretic hormone antagonists), natriuretic agents, or agents that otherwise offload volume and clear solutes from the body.

One aspect of the invention provides improved devices and methods for treating acute kidney injury, acute kidney disease, and chronic kidney disease.

Another aspect of the invention provides devices and methods for treating acutely decompensated heart failure, and/or chronic heart failure.

One embodiment includes a vacuum pump with a pressure check valve that ensures a desired vacuum pressure can be established in the renal pelvis. The valve could be electronically controlled or proportional to modulate pressure depending on therapeutic targets.

Another embodiment includes a vacuum pump with a pressure sensor and feedback loop that ensures a desired vacuum pressure can be established in the renal pelvis.

Another embodiment provides a nephrostomy and ureter-occluding vacuum catheter for use in treating the above-mentioned conditions.

One embodiment uses a multi lumen balloon catheter to occlude the ureter while pulling a vacuum to draw fluid into the catheter and deliver it to the bladder.

In one variation, the pump used to create the vacuum may be implanted subcutaneously. The pump could have an integrated or connected pressure sensor (measuring mechanical or oncotic pressure) that regulates vacuum to a set level.

One aspect of the invention is a miniaturized pump that is implantable directly in the renal pelvis or ureter. In one embodiment this pump has built in pressure sensors. This pump may be subcutaneously powered or charged. The method of power transfer could be inductive or magnetic, for example.

In one embodiment the pump can be programed to different pressure routines or curve profiles.

In one embodiment a pump is implantable in the bladder, allowing a larger pump size. A bladder implanted pump could be powered or could be manually operated through the abdomen. Conceivably, the pump is also implantable in the peritoneal cavity of the abdominal or retroperitoneal wall and could be manually powered or transcutaneously powered through the derma.

In one embodiment a lower pressure environment could be created within the renal capsule, rather than the renal pelvis. This embodiment could be combined with an injection of an ADH-antagonist/diuretic/dialysate inside the renal capsule.

In one embodiment, a dual nephrostomy system is provided that may alternate between a vacuum and a liquid infusion.

One aspect of the invention provides an infusion/suction catheter with one or more balloons attached thereto for occluding the ureter and controlling flow through the balloon with a pump. Controlling the flow could involve alternating between suction and infusion.

In one embodiment, an infusion/suction catheter is provided with a sample port used to take urine samples during a procedure to perform tonicity or other laboratory tests. The catheter or the sample port could also be used to inject medications or drug instillations.

One aspect of the invention is a method for improving kidney function that includes creating a vacuum within a renal pelvis of a kidney of a patient, thereby drawing urine out of renal tissue surrounding the renal pelvis. Creating the vacuum within the renal pelvis may be accomplished by introducing a suction catheter into the renal pelvis.

In one embodiment, this method further involves occluding the ureter prior to creating the vacuum.

In this or another embodiment, the method further involves directing urine from the renal pelvis to the bladder.

In this or another embodiment, creating the vacuum within the renal pelvis is accomplished by placing a one way valve within a ureter that prevents retrograde flow from a bladder toward the kidney; slowly inflating a balloon within the renal pelvis, thereby reducing a volume within the renal pelvis thus displacing urine contained therein through the valve and toward the bladder; and rapidly deflating the balloon, thereby increasing a volume in the renal pelvis and thus creating a temporary vacuum within the renal pelvis until the urine drawn out of the renal tissue surrounding the renal pelvis refills the renal pelvis.

In another aspect of the invention, creating the vacuum within the renal pelvis includes occluding a ureter associated with the kidney; and using a pump to remove fluid from the renal pelvis. The method may also include directing the fluid removed from the renal pelvis to a bladder through the ureter.

One aspect of the invention is a system for improving kidney function of a patient that includes a catheter having a proximal end and a distal end; a pump connected to one of the ends; and a fluid control mechanism that directs fluid manipulated by the pump out a renal pelvis of the kidney such that a vacuum is established in the renal pelvis resulting in increased fluid production by the kidney.

In some embodiments, the pump is connected to the proximal end. In other embodiments, the pump may be a balloon connected to the distal end. In still other embodiments, the pump may be a propeller pump contained within a distal end of the catheter. The pump may be external to the patient or may be implantable within the patient.

The fluid control mechanism may include holes formed in a sidewall of the catheter.

One aspect of the invention is a system for improving kidney function in a patient that includes a pump; a first nephrostomy tube connected at a proximal end to the pump and having a distal end suited for placement in a renal pelvis of a first kidney of the patient; and a second nephrostomy tube connected at a proximal end to the pump and having a distal end suited for placement in a renal pelvis of a second kidney of the patient; wherein when said first and second nephrostomy tubes are placed in the renal pelvises of the first and second kidneys of the patient, activating the pump creates a negative pressure in the renal pelvises. The pump system may also include a reservoir or a port that can be attached to a reservoir such that the pump may infuse a therapeutic agent.

At least one of the embodiments of this system also includes a catheter connected at a proximal end to the pump and having a distal end suited for placement in a ureter associated with one of the kidneys, and wherein said pump is configured to direct fluid from the first and second nephrostomy tubes into the catheter such that the fluid is delivered to a bladder of the patient.

At least one of the embodiments of this system also includes a container connected to the first and second nephrostomy tubes distal of the pump such that fluid drawn toward the pump through the first and second nephrostomy tubes is deposited in the container. In at least one embodiment, the container is a bag.

At least one of the embodiments of this system also includes a port usable to inject therapeutic fluids into the renal pelvis.

At least one of the embodiments of this system also includes a sample port in at least one of the first and second nephrostomy tubes for use in taking urine samples.

One aspect of the invention is a method of improving kidney function in a patient comprising: reducing pressure in a renal system of a least one kidney of a patient; infusing a therapeutic agent into the renal system of the at least one kidney; said reducing pressure and said infusing a therapeutic agent being conducted in combination during a therapeutic intervention on said patient.

Another aspect of the invention is a method of improving kidney function in a patient comprising: creating an access route to a renal pelvis region of a kidney of said patient through a wall of said kidney; drawing a vacuum through said access route so as to increase kidney function as a result of said vacuum.

Another aspect of the invention is a method of improving kidney function of a patient comprising: creating access to a renal pelvis region of a kidney of said patient through a wall of said kidney; introducing a therapeutic agent into said pelvis region so as to increase kidney function as a result of said introduction of said therapeutic agent.

Yet another aspect of the invention is a system for improving kidney function of a patient comprising: an access device configured for placement through a wall of a kidney to a renal pelvis region of said kidney; a vacuum inducing component associated with said access device configured for drawing a vacuum in said renal pelvis region; an infusion component associated with said access device configured for introducing a therapeutic agent into said renal pelvis region; said access device, said vacuum inducing component and said infusion inducing component in combination, during operation, increasing kidney function.

One aspect of the invention is a system for improving kidney function of a patient comprising: an access device configured for placement through a wall of a kidney to a renal pelvis region of said kidney; a vacuum component associated with said access device configured for drawing a vacuum in said renal pelvis region; said access device and said vacuum inducing component, in operation, increasing kidney function.

Still another aspect of the invention is a system for improving kidney function of a patient comprising: an access device configured for placement through a wall of a kidney to a renal pelvis region of said kidney; an infusion component associated with said access device configured for introducing a therapeutic agent into said renal pelvis region; said access device and said infusion inducing component in combination, in operation, increasing kidney function.

Another aspect of the invention is a method of improving kidney function in a patient comprising: inserting a catheter percutaneously into a renal pelvis region of a kidney; drawing a vacuum through said catheter so as to increase kidney function as a result of said vacuum.

Yet another aspect of the invention is a system for improving kidney function of a patient comprising: a percutaneous access device configured for placement in a renal pelvis region of said kidney; a vacuum component associated with said access device configured for drawing a vacuum in said renal pelvis region; said access device and said vacuum inducing component, in operation, increasing kidney function.

Still another aspect of the invention is a system for improving kidney function of a patient comprising: a percutaneous access device configured for placement in a renal pelvis region of said kidney; an infusion component associated with said access device configured for introducing a therapeutic agent into said renal pelvis region; said access device and said infusion inducing component in combination, in operation, increasing kidney function.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a diagram of an embodiment of a catheter system of the invention placed within a kidney being treated;

FIG. 2 is a diagram of an embodiment of a catheter system of the invention placed within a kidney being treated;

FIG. 3 is a diagram of an embodiment of a catheter system of the invention placed within a kidney being treated;

FIG. 4 is a diagram of an embodiment of a catheter system of the invention placed within a kidney being treated;

FIG. 5 is an example of a display or user interface embodiment for use with the systems of the invention;

FIG. 6 is a diagram of an embodiment of a catheter system of the invention placed within a kidney being treated;

FIG. 7 is a diagram of an embodiment of a catheter system of the invention placed within a kidney being treated;

FIG. 8 is a diagram of an embodiment of a catheter system of the invention placed within a kidney being treated;

FIG. 9 is a diagram of an embodiment of a catheter system of the invention placed within a kidney being treated;

FIG. 10 is a diagram of an embodiment of a catheter system of the invention placed within a kidney being treated;

FIG. 11 is a diagram of an embodiment of a catheter system of the invention placed within a kidney being treated;

FIG. 12 is a diagram of an embodiment of a pump useable with one or more of the catheter system embodiments of the invention;

FIG. 13A is a diagram of an embodiment of an implantable pump system of the invention implanted in a kidney being treated;

FIG. 13B is a diagram of the internal components of the embodiment of the implantable pump of FIG. 13A;

FIG. 14 is a diagram of an embodiment of a pump useable with one or more of the catheter system embodiments of the invention;

FIG. 15 is a diagram of an embodiment of a pump useable with one or more of the catheter system embodiments of the invention;

FIG. 16A is a diagram of an embodiment of a pump useable with one or more of the catheter system embodiments of the invention;

FIG. 16B is a close-up view of the impeller assembly of the pump of FIG. 16A;

FIG. 16C is a close-up perspective view of the impeller assembly of the pump of FIG. 16A;

FIG. 16D is a close-up elevation view of the impeller assembly of the pump of FIG. 16A;

FIG. 16E is an embodiment of a propeller usable with the impeller assembly of the pump of FIG. 16A;

FIG. 16F is an embodiment of a propeller usable with the impeller assembly of the pump of FIG. 16A;

FIG. 17 is a diagram of an embodiment of a pump useable with one or more of the catheter system embodiments of the invention;

FIG. 18 is a diagram used to illustrate an embodiment of a method of the invention;

FIG. 19 is a diagram used to illustrate an embodiment of a method of the invention;

FIG. 20 is a diagram used to illustrate an embodiment of a method of the invention;

FIG. 21 is a diagram used to illustrate an embodiment of a method of the invention;

FIG. 22 is a diagram used to illustrate an embodiment of a method of the invention;

FIG. 23 is a diagram used to illustrate an embodiment of a method of the invention;

FIG. 24 is a diagram used to illustrate an embodiment of a method of the invention; and,

FIG. 25 is a diagram used to illustrate an embodiment of a method of the invention;

FIG. 26 is a diagram used to illustrate an embodiment of a method of the invention;

FIG. 27 is a diagram used to illustrate an embodiment of a method of the invention;

FIG. 28 is a cross-section of an embodiment of a multi-lumen catheter usable with the invention; and,

FIG. 29 is an anatomical diagram showing an access point between the femoral artery and a ureter of a patient undergoing an embodiment of a method of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

In general, the present invention is directed to improving kidney function by controlling the flow into and out of the kidneys, either through the ureters or through a catheter, the general urine collection system, or all. The invention involves several embodiments of devices, systems of devices, and methods for using these systems and devices. From the most general perspective, the invention improves kidney function by inducing a negative pressure in the urine collecting system, provides a means of infusing therapeutic agents that induce polyurea and/or solute clearance, or combines these two methods to induce a multiplicative effect. For purposes of organization, the description of the invention will be broken into catheters, pumps and methods. It is to be understood that every catheter, pump and method can incorporate any of the other components.

Catheter Systems

The catheter systems described herein may be inserted using several percutaneous or non-percutaneous approaches. As used herein, percutaneous approaches involve puncturing the skin of the patient to provide an access point for the catheter. Non-percutaneous approaches do not puncture the skin, and would thus involve routing a catheter through the urethra, bladder, ureters and into the kidney.

One example of a percutaneous approach is a nephrostomy approach in which an artificial opening or stoma is created between the kidney and the skin, which allows for a urinary diversion directly from the upper part of the urinary system, namely the kidneys and/or the ureters. The nephrostomy may be temporary or may include the installation of a semi-permanent or permanent port for use with embodiments of the present invention or with known treatment methods. The catheter systems may also be inserted using a retrograde ureteral approach. Lastly, the renal pelvis may be accessed using a femoral venous approach and trans-ureteral puncture, using a snare, magnet, or other targeting system.

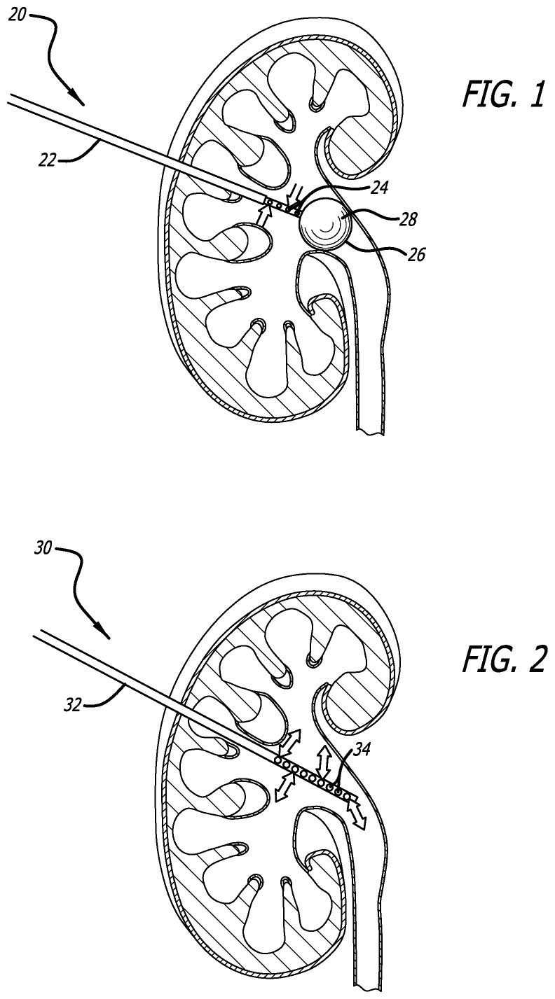

Referring to FIG. 1, there is shown a balloon catheter assembly 20 that includes a catheter 22 having a plurality of holes 24 leading to a central lumen (not shown). At a distal end 26 of the catheter 22 is an occlusion balloon 28. When inflated, the balloon 28 blocks the ureter, allowing the catheter 22 to be used to draw a vacuum in the renal pelvis through the holes 24. The holes 24 can also be used to perfuse the urine collection system with therapeutic agents. In at least one embodiment, this catheter system is used in conjunction with a reversible pump or multiple pumps such that the holes 24 may be used to alternate between drawing a suction and perfusing a therapeutic agent.

FIG. 2 shows a catheter assembly 30 that includes a catheter 32 having a plurality of holes 34 leading to a central lumen. The holes 34 serve as vacuum holes so that when the catheter is placed across the renal pelvis and into the ureter, as shown, the vacuum collapses the ureter onto the catheter 32 while simultaneously pulling a vacuum in the renal pelvis. In at least one embodiment the central lumen may be bifurcated into a suction lumen and a perfusion lumen. The suction lumen may lead to the distal most holes that are placed in the ureter such that, when they draw a suction, the ureter collapses around the catheter 32. The proximal holes that are placed in the renal pelvis may be in communication with the perfusion lumen such that therapeutic agent may be introduced at the same time suction is being applied to the distal most holes. There may be another lumen that leads directly to a distal end used for directing urine into the ureter.

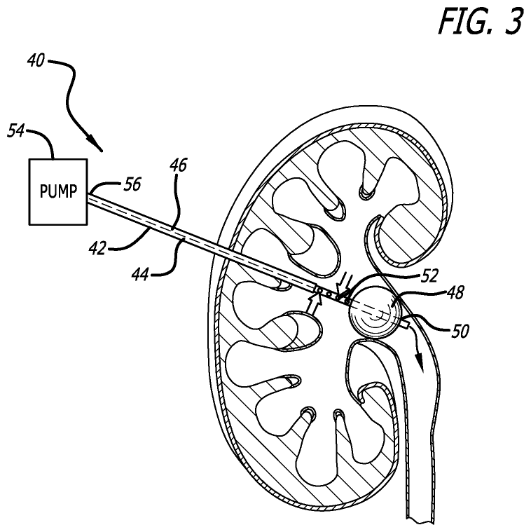

FIG. 3 shows a multi-lumen balloon catheter assembly 40 that includes a catheter 42 having at least a first lumen 44 and a second lumen 46 that passes through an occlusion balloon 48 at a distal end 50 of the catheter 42. The second lumen 46 opens at the distal end 50 of the balloon 48. The catheter 42 includes a plurality of holes 52 leading into the first lumen 44. The catheter may include a third lumen for infusing therapeutic agents.

The catheter assembly 40 further includes a pump 54 connected to a proximal end 56 of the catheter 42 and is attached such that the pump may be used to pull a vacuum in the renal pelvis through the first lumen 44, which is in communication with the renal pelvis via the holes 52. Additionally, the pump may be also connected to the second lumen 46 so that it may pump the urine from the renal cavity back through the second lumen 46 into the ureter so the urine may enter the bladder. The pump may have a reservoir (not shown) or be connected to a reservoir to perfuse the renal pelvis with therapeutic agents through the holes 52.

In this embodiment, a reciprocating pump may be used to draw urine proximally from the renal pelvis during one half cycle of the reciprocating pump and push the urine through a second lumen during the other half of the reciprocating pump cycle. One or more check valves may be employed to prevent retrograde flow through the lumens. Alternatively, two pumps may be employed, a vacuum pump associated with a first lumen 44, and a positive pressure pump associated with the second lumen 46. A controller, switching system, clock, or other mechanism may be used to synchronize the two pumps. Alternatively, both pumps could be non-positive displacement pumps that run continuously. Other pump embodiments include peristaltic or impeller pumps. These could also be reversible.

The embodiments of FIGS. 2 and 3 have a chronic application, where the pump can be implanted subcutaneously. The pump could have an integrated or connected pressure sensor that regulates a vacuum to a set level.

It is also envisioned that the first lumen 44 could be used for both suction and perfusion of a therapeutic agent, while the second lumen 46 could be used simultaneously, sequentially, or alternatingly for discharge of urine into the bladder. This embodiment may be accomplished with a reciprocating pump aligned with the first lumen, and a non-positive displacement pump aligned with the second lumen. Alternatively, a single, reciprocating pump could be used that draws suction through the first lumen during one half cycle, and during the other half cycle pumps therapeutic agent through the first lumen while simultaneously pumping urine through the second lumen.

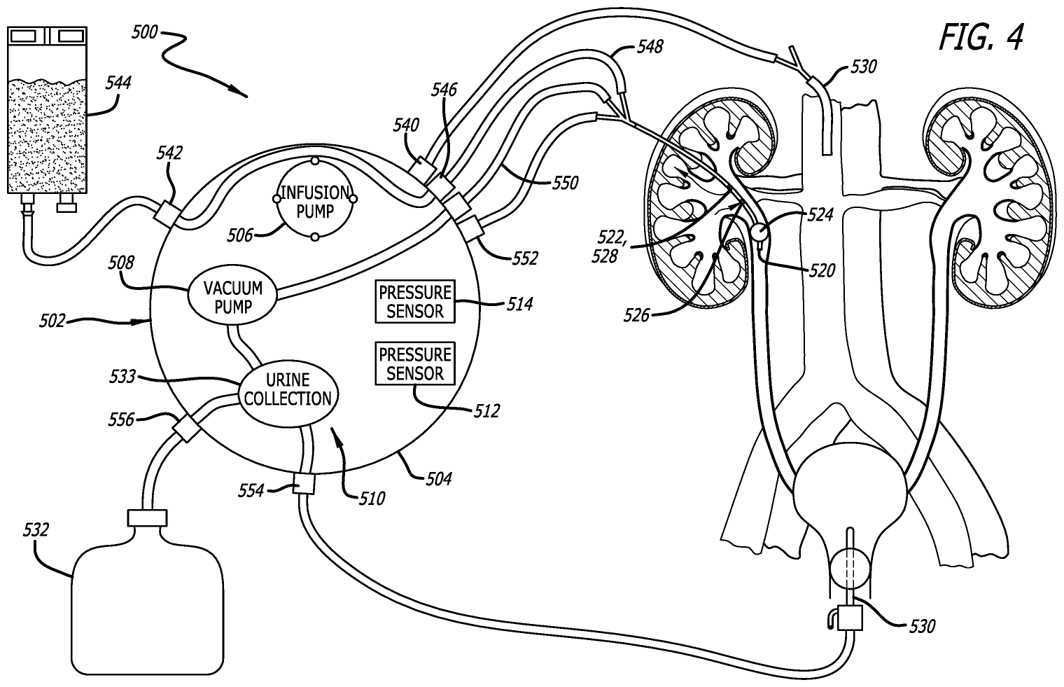

FIG. 4 depicts a catheter system 500 of the invention that allows a therapeutic regimen that intermittently perfuses a therapeutic agent or agents and applies a vacuum to the urine collecting system. The system 500 includes a fluid management assembly 502 that includes a housing that may serve as a handle 504, an infusion pump 506, a vacuum pump 508, a urine collection system 510 as well as pressure sensors 512 and 514 that take reading from a therapy catheter 520 and a central venous catheter 530. The therapy catheter 520 include port holes 522 at a distal end thereof, just proximal of an isolation or sealing element 524, shown as a balloon by way of example. The catheter 520 may include both infusion and vacuum lumens 526 and 528, respectively. The urine collection system 510 may include a foley catheter 530 routed to the bladder through the urethra, and a urine collection reservoir 532 connected to the foley catheter 530 via a urine routing chamber 533. The urine routing chamber 533 is connected to the vacuum pump 508 such that urine may be drawn from the bladder and into the urine collection reservoir 532 as shown. The vacuum pump draws a vacuum at an elevated location on the urine routing chamber 533 such that when urine enters the chamber 533, gravity draws it downward, preventing it from entering the vacuum pump. The urine collection reservoir is connected at a low point on the routing chamber 533 such that the urine drains into the reservoir 532.

The fluid management assembly 502 further includes connectors or ports for connecting the catheters and other fluid lines to the components of the fluid management assembly 502. For example, a first connector 540 attaches the central venous catheter to the pressure sensor 514. A second connector 542 acts as an input for connecting a fluid agent supply 544 to the infusion pump 506. A third connector 546 is an output port that connects the infusion pump to an infusion line 548 that is connectable to the therapy catheter 520. A fourth connector 550 connects the vacuum pump 508 to the therapy catheter 520. A fifth connector 552 connects the therapy catheter to the pressure sensor 512. A sixth connector 554 connects the foley catheter 530 to the vacuum pump 508. Alternatively, gravity could be used. A seventh connector 556 connects the urine routing chamber 533 to the urine collection reservoir 532.

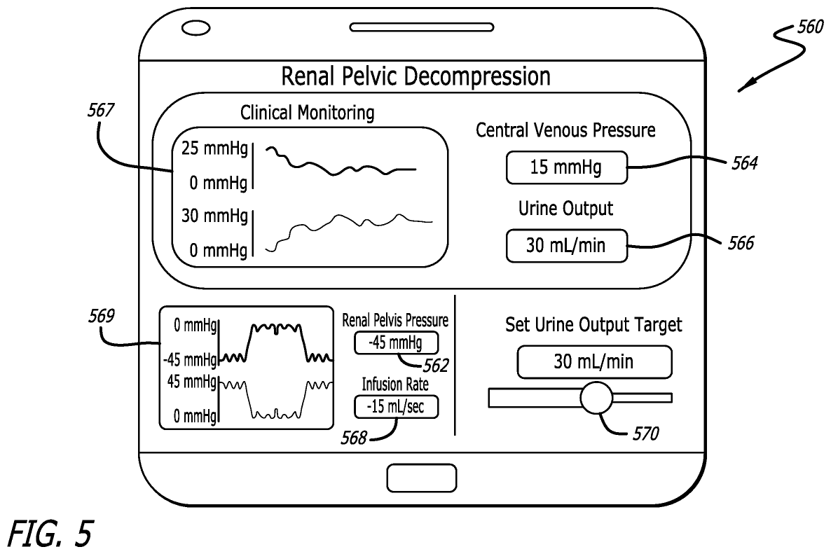

The fluid management assembly 502 may further include a display 560, depicted in FIG. 5, that provides data fields 562 and 564 provided by the sensors 512 and 514 as well as data fields 566 and 568 for measured urine output and infusion rate. Graphical displays 567 and 569 of pressure over time, urine output over time, pelvis pressure over time, and infusion rate over time. A control 570 is also provided that sets a target urine output. A target central venous pressure may also be set (not shown). The assembly 502 may be programmed such that increasing the target output may automatically increasing the speeds, duty cycle, or duration, for example, of one or both of the suction pump and the infusion pump. It is envisioned that the fluid management assembly 502 would have further sensors and capabilities, such as a urine off total, various flow rates, integration with an electronic medical record, MRI compatibility, and the like.

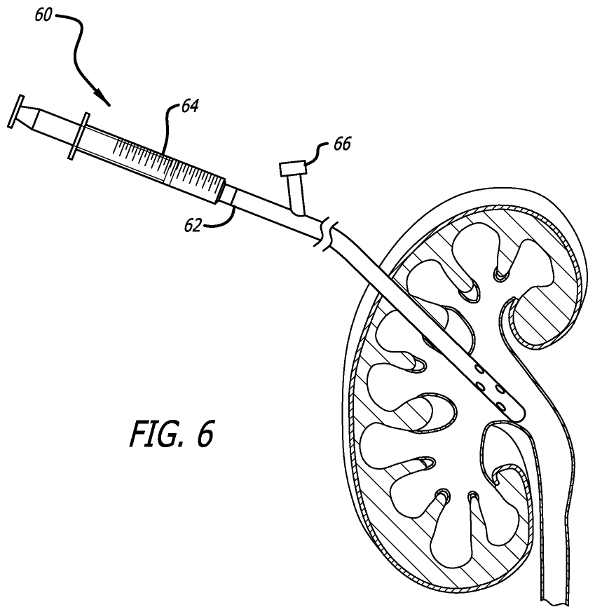

FIG. 6 depicts a diagram of a port feature 60, which can be incorporated into any of the catheter assemblies described herein, that provides a first port 62 to which a syringe 64 may be attached for introducing medications or other fluids into the kidney. This first port 62 may also be attached to a pump embodiment, as previously described. Additionally, a sample port 66 may be provided that is used to take urine samples to test the tonicity of the urine, assessing the efficacy of the treatment, sampling the sodium content of the urine, Sample the sodium content of the urine, assessing creatinine, inulin, and/or general solute clearance, assessing glomerular filtration rate, and the like.

This embodiment may be used with one or multiple lumens. The use of multiple lumens may be desirable if samples are being taken after the introduction of medication or other fluids to prevent contamination of the urine samples.

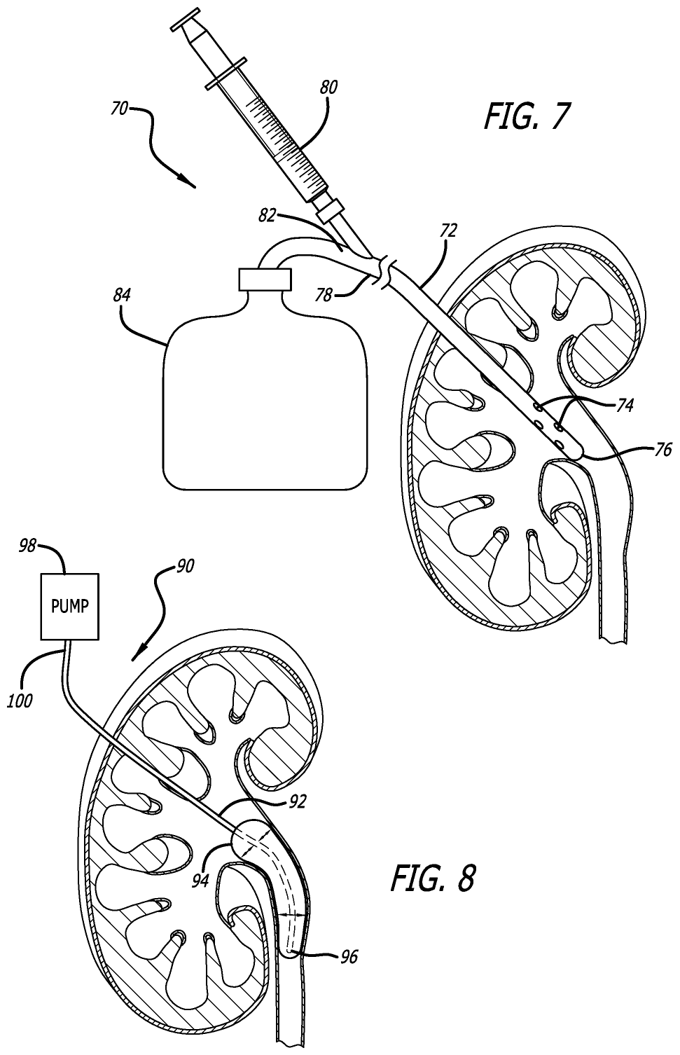

FIG. 7 shows an embodiment of a catheter system 70 that can be used to manually induce a negative pressure in the renal pelvis. The system 70 includes a catheter 72 with multiple fenestrations or ports 74 at a distal end 76 thereof, useable to suck renal fluid into the catheter when a negative pressure is created at a proximal end 78 of the catheter 72. The negative pressure may be created with a syringe 80, as shown, or another form of a powered or manual pump. The catheter 72 includes a branch 82 that is connected to a urine collection bag or container 84. The branch 82 and container 84 are oriented such that, when the syringe is used to create a vacuum in the catheter 72, urine flows out of the renal cavity and drops into the container or bag 84, due to gravity, instead of entering the syringe 72. It is conceivable that this embodiment could also be used to pump infusate into the renal cavity. A valve, manually operated or otherwise, could be placed at the neck of the bag 84 to close the bag while the syringe 80 is used to introduce a therapeutic agent into the renal cavity.

FIG. 8 shows an embodiment of a balloon catheter system 90 that includes a catheter 92 with a balloon 94 attached to a distal end 96 of the catheter 92. A fluid pump 98 is attached to a proximal end 100 of the catheter 92 that is able to slowly inflate and rapidly deflate the balloon 94 such that the flow of fluid through the ureter is assisted or enhanced via the creation of a temporary vacuum. The catheter 92 is positioned such that the balloon 94 is located within the ureter. The balloon, in another embodiment, could perceivably be placed in the renal pelvis. When the balloon 94 is inflated, pressure fails to build on the kidney or upstream side of the balloon 94 due to the relatively slow nature of the inflation. When the balloon is rapidly deflated, the occupied volume is quickly released and the flow through the ureter is greater than a steady state flow that would occur if the balloon were not inflated. As it is shown in FIG. 8, the catheter is routed through the renal capsule, past the renal pelvis, and into the ureter. It is conceivable that the catheter of this embodiment could be routed through the urethra, bladder, and into the ureter or renal pelvis.

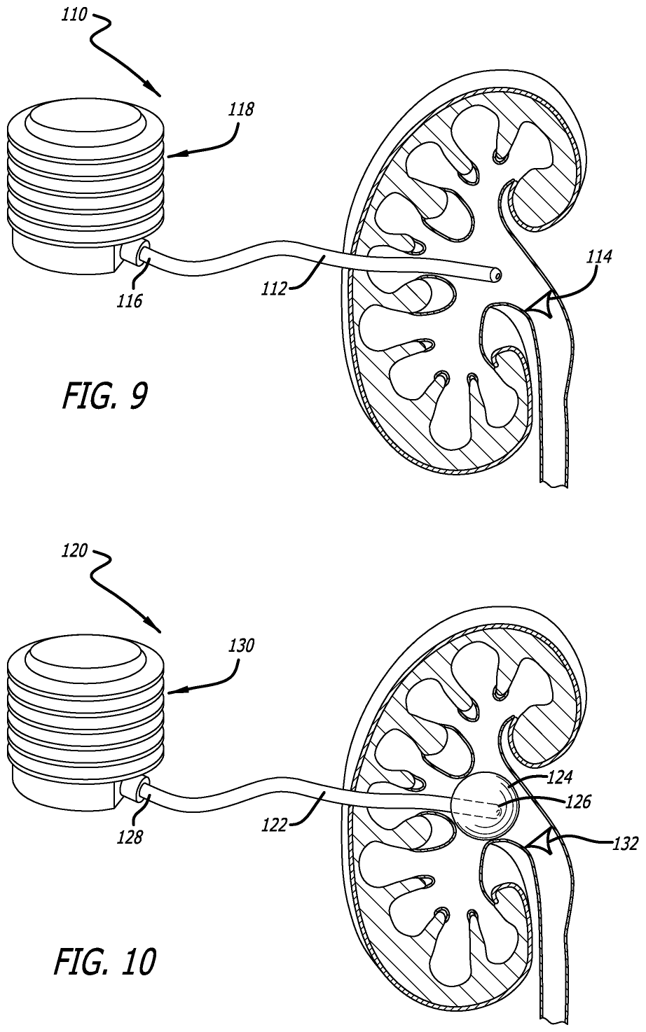

FIG. 9 shows an embodiment of a catheter system 110 including a catheter 112 that operates in conjunction with a check valve 114 placed in the ureter. The proximal end 116 of the catheter 112 is attached to a pump 118. The embodiment of the pump 118 shown is a manual, fluid-filled, spring-open pump that may be implanted subcutaneously and operated by the user. The one-way or check valve 114 is placed in the ureteropelvic junction. When the pump 118 is compressed, fluid contained in the pump 118 is driven through the catheter 112 and into the renal pelvis where it forces the fluid already contained within the renal pelvis through the check valve 114. Spring force within the pump 118 then begins bringing the pump 118 to a pre-compressed state, which creates negative pressure within the renal pelvis. This draws urine from the outer components of the kidney into the renal pelvis, thus improving kidney function. As urine flows into the pelvis, some of the urine will travel into the pump 118 until the pump is full and the pressure in the renal pelvis is equalized. The user may then recompress the pump 118. It is also perceivable that the actuation of the pump is automatically cycled to match a physiologic signal, such as pressure in the renal pelvis, central venous pressure, urine output, etc.

FIG. 10 shows an embodiment of a catheter system 120 that is similar to catheter system 110 except that it includes a catheter 122 with a balloon 124 at a distal end 126 of the catheter 122. The proximal end 128 of the catheter is attached to a pump 130 similar to pump 118 of the embodiment 110 of FIG. 9. A check valve 132 is placed in the ureteropelvic junction. When the pump 130 is compressed, fluid contained in the pump 130 is driven through the catheter 122 and into the balloon 124, causing the balloon to inflate and displace urine in the renal pelvis. The urine is forced through the check valve 132 and into the ureter where it continues to the bladder. Spring force within the pump 130 then begins bringing the pump 130 to a pre-compressed state, sucking the fluid out of the balloon 124. The decrease in balloon size creates negative pressure within the renal pelvis. This draws urine from the outer components of the kidney into the renal pelvis, thus improving kidney function. As urine flows into the pelvis, all of the fluid will travel into the pump 130 from the balloon 124 until the pump is full and the pressure in the renal pelvis is equalized. The user may then recompress the pump 130. It is also perceivable that the actuation of the pump is automatically cycled to match a physiologic signal, such as pressure in the renal pelvis, central venous pressure, urine output, etc.

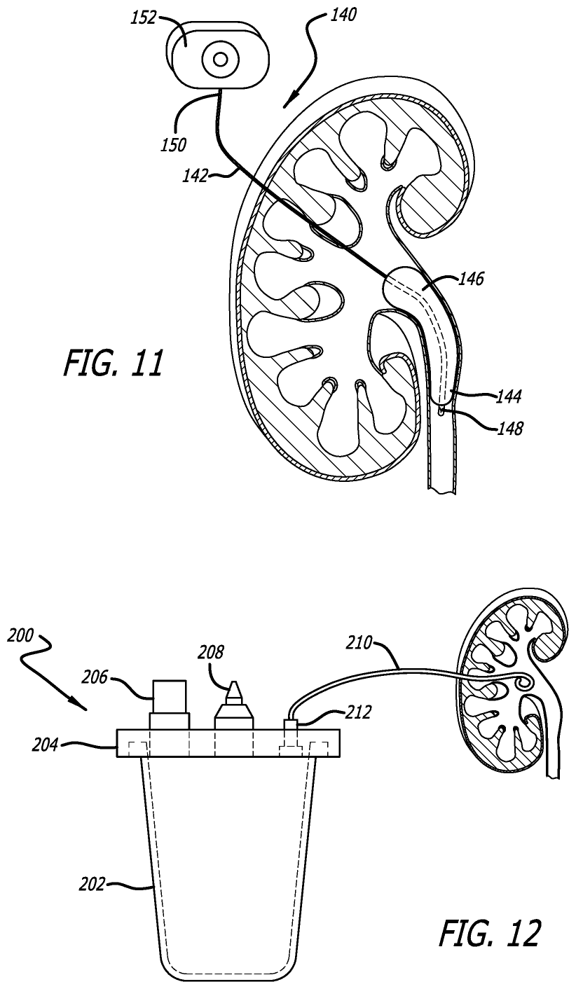

FIG. 11 depicts a catheter system 140 that includes a small-diameter catheter 142 that has a distal end 144 that includes a balloon 146 that is placed in the ureter. The distal end of the catheter 140 extends through the balloon 146 and includes a valve 148, such as a bi-leaflet or duckbill valve, that prevents retrograde flow through the catheter. The proximal end 150 of the catheter 142 is connected to a compressible pump 152. Using the pump to inflate the balloon 146 creates a pumping action similar in operation to the system of FIG. 10. The pump 152 shown is an implantable, nitinol-reinforced compressible vacuum generator with a saline/drug infusion capability.

Pumps

Referring now to FIG. 12, there is shown an embodiment 200 of a pump assembly that includes a rigid container 202, preferably graduated, with a rigid, removable lid 204. The lid 204 includes a vacuum pump 206, a check valve 208 and a drainage catheter 210 or a port 212 to which a drainage catheter 210 may be attached. The check valve 208 is optional but acts as a safety feature that allows a set pressure to be maintained inside the container. The check valve 208 opens when the pressure inside the container 202 exceeds the set pressure and allows air or another selected gas to enter the container 202 until the set pressure is reestablished.

Alternatively, the vacuum pump 206 could have an integrated pressure sensor and feedback loop that allows the vacuum pressure to be automatically regulated. In one embodiment, the feedback loop is programmable such that various pressure profile curves may be entered and followed. In another embodiment, the check valve 208 may be controlled with a programmable motor such that various pressure profile curves or variances may be entered and followed.

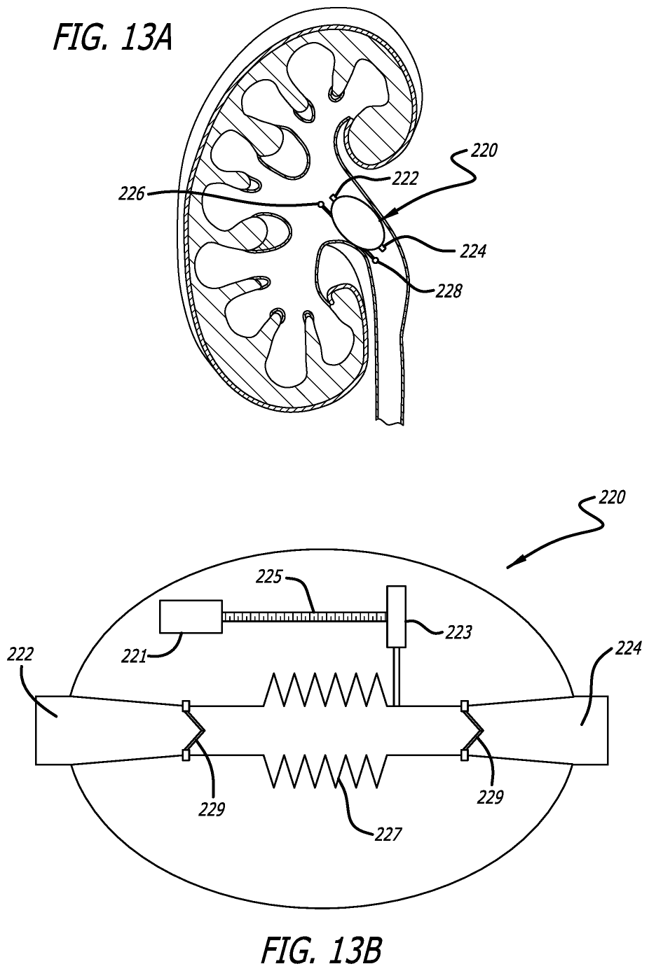

FIGS. 13A and 13B show an embodiment of an implantable pump 220. The pump 220 may be a miniaturized version of any of the non-manual embodiments of the pumps described herein. The pump 220 may be implanted in the renal pelvis, at the ostium to the ureter. In one embodiment, the pump may be implanted in the ureter. In one embodiment, the pump includes an inflatable balloon or resilient balloon that anchors the balloon at the ostium to the ureter and creates a seal.

The pump 220 may configured with built in pressure sensors. For example, the pump may have an inlet side 222 oriented in the renal pelvis and an outlet or discharge side 224 oriented facing, or located within, the ureter. A first pressure sensor 226 may be located on the inlet side 222 and a second pressure sensor 228 may be located on the outlet side 224. Having pressure sensor on either side of the pump 220 allows a differential pressure across the pump to be determined, thereby allowing a determination of flow rate.

The pump 220 may be subcutaneously powered or charged. In one embodiment, the pump 220 may be charged using wireless charging technology. Further, the pump 220 may be programmed to different pressure routines. Alternatively, the pump 220 could be designed for placement in the bladder, and have tubing extending into the renal pelvis. A bladder version of the pump 220 could be much larger as the bladder has more room than the renal pelvis.

FIG. 13B shows one embodiment of the internal mechanisms of the pump 220. In this embodiment, the pump 220 is powered by a motor 221 that drives a travel nut 223 with a lead screw 225 in, for example, reciprocating fashion. The travel nut 223 is attached to a bellows 227 that is expanded and contracted by the axial movement of the travel nut 223, thus pumping fluid through the inflow 222 and outflow 224 via check valves 229.

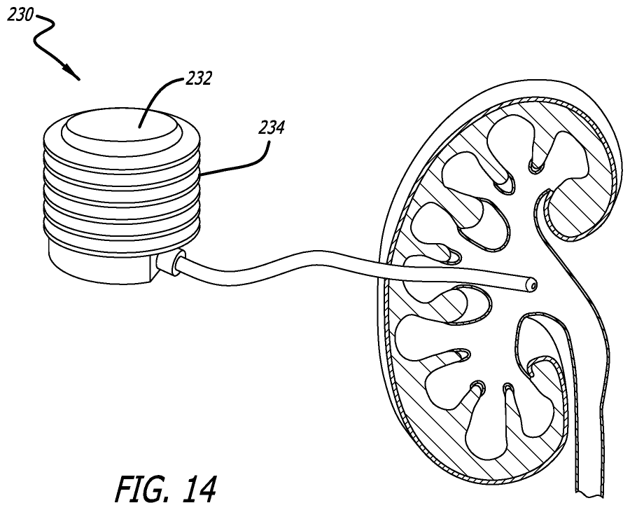

FIG. 14 shows an embodiment of a manual pump 230 that includes a compressible chamber 232 that returns to a restored state with spring force. The spring force may be provided by a spring located within or around the chamber 232, or as shown in FIG. 14, the chamber 232 may include pleated bellows 234 that resist being compressed and return to an expanded state when released.

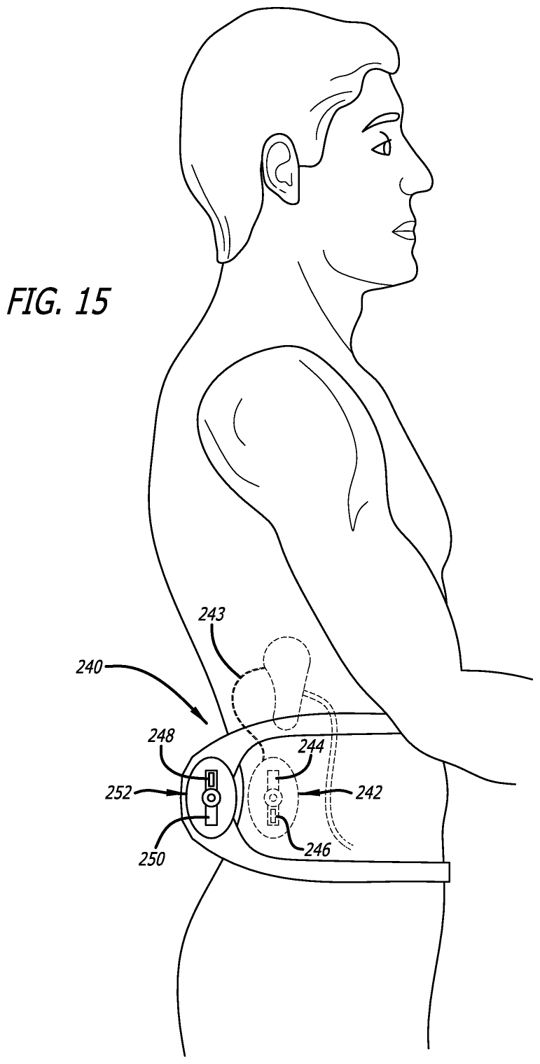

FIG. 15 shows a magnetically-driven, implantable pump 240 that includes a rotatable implantable impeller 242 that drives fluid from the renal pelvis towards the urinary outflow tract through a catheter 243 that has at least one terminus in a kidney. The impeller 242 has magnetic north 244 and south 246 poles that are respectively attracted to south 248 and north 250 poles of an electrically-powered drive impeller 252. The drive impeller is, in one embodiment, worn on the body, such as on a belt, near the skin and proximal the implantation site of the implanted impeller 242. The drive impeller 252 may be battery-powered.

In one embodiment the pump impeller 242 is implanted next to the kidney or subcutaneously and attached to a renal catheter such as that shown in FIG. 3, for example. The internal component 242 of the pump 240 does not require electronics.

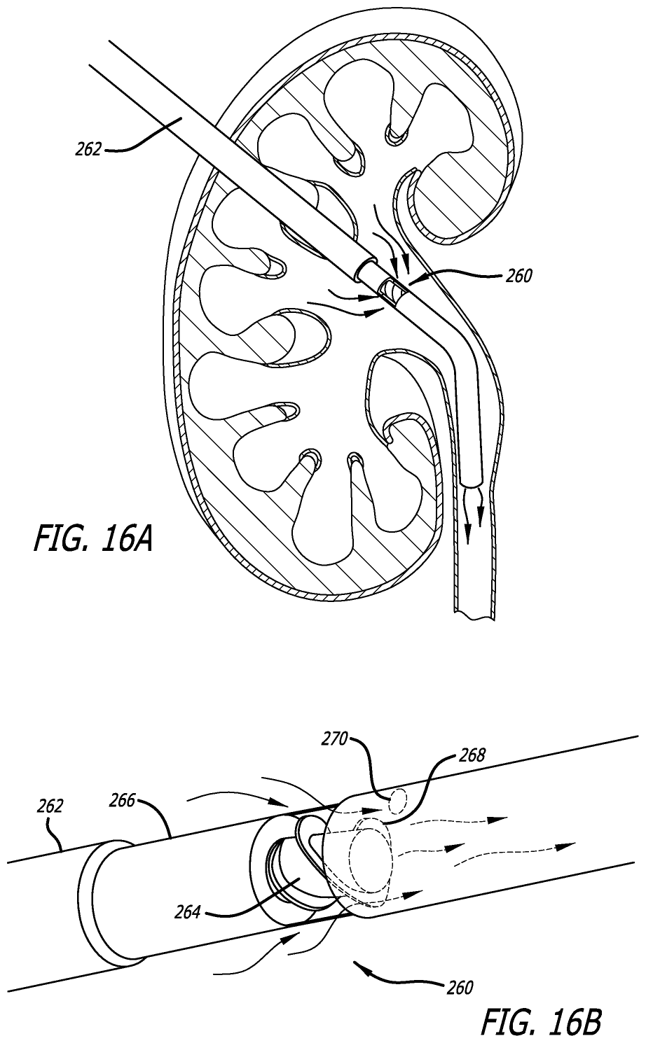

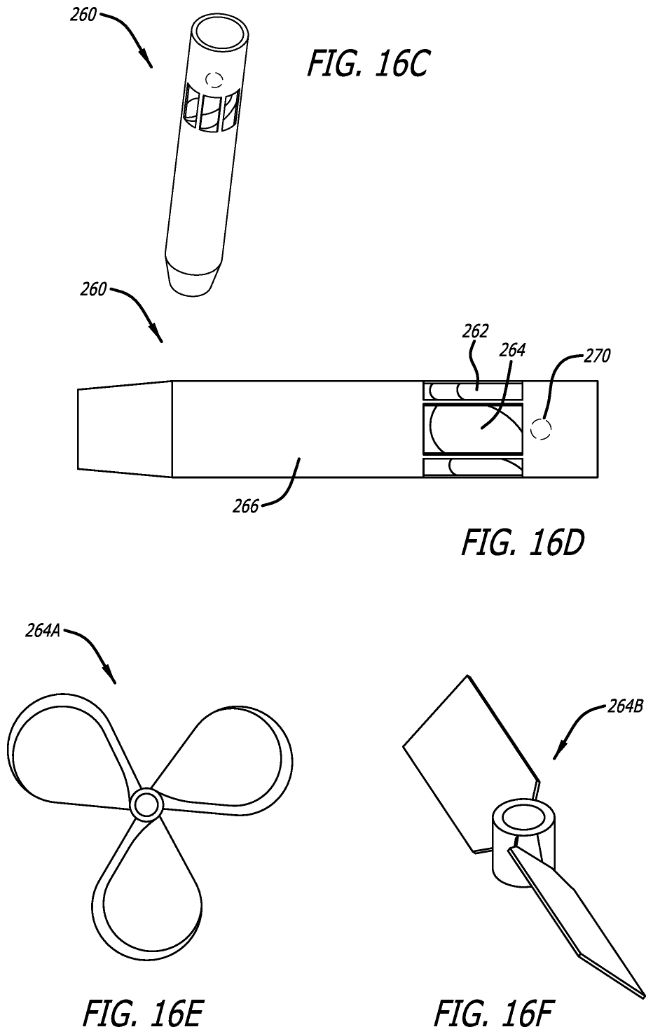

FIGS. 16A through 16D depict an axial flow impeller 260 that resides within an impeller catheter 262. The axial flow impeller 260 includes an impeller 264, shown in FIG. 16B, that is driven by a motor 266. A gap in the catheter 262 is located between the motor 266 and the blades 268 of the impeller 264, forming a urine inlet where urine is drawn into the catheter by the negative pressure created on the inlet side of the impeller. The axial flow impeller 260 may further include an optical sensor 270 which allows feedback control of the pump speed and associated vacuum or infusion level.

FIGS. 16E and 16F show two examples of impellers 264A and 264B, respectively. FIG. 16E shows a three-bladed propeller. By way of convention, as used herein, a propeller has an open design whereas an impeller is in a casing or catheter and is used to move water through the catheter. As such, when the propeller shown in FIG. 16E is placed within the pump catheter 262, it becomes an impeller.

FIG. 16F shows an example of a four-bladed 45-degree pitched blade turbine. The devices of 16E and 16F are just examples of impeller/propeller designs useable with the pumps of the invention and are not to be interpreted as limiting examples.

The axial flow impeller 260 may be fully implantable, subcutaneous or intra-renal. For example, this axial flow impeller 260, including its power source and drive train, could be implanted subcutaneously and charged via induction. The impeller 260 could also be placed in the retrograde ureteral fashion with no percutaneous access.

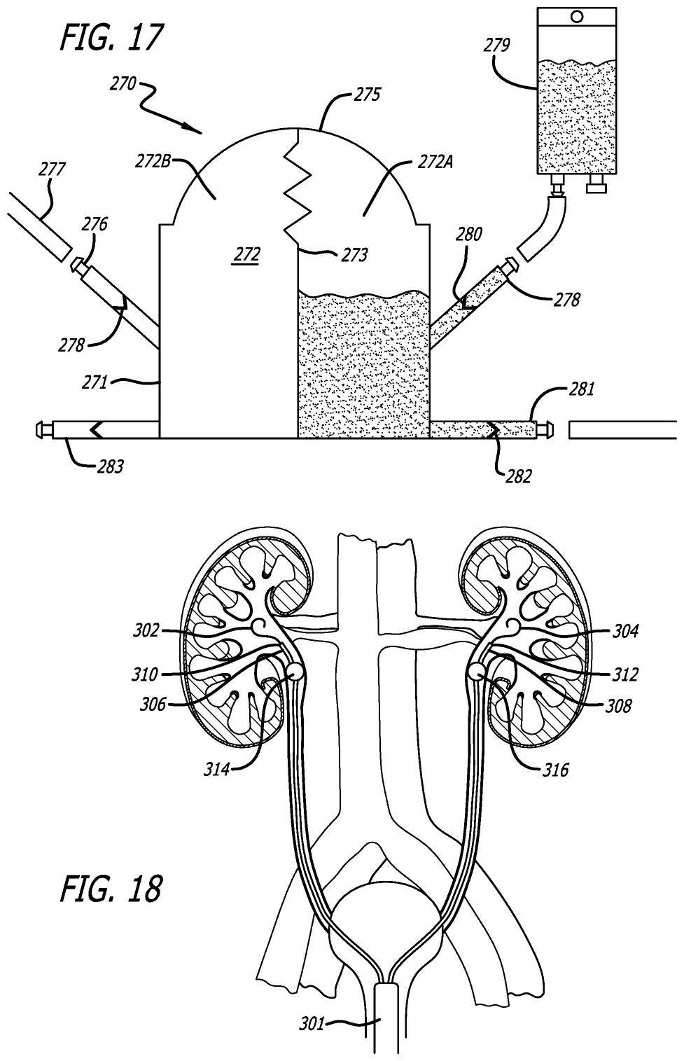

Another pump system 270 of the invention is shown in FIG. 17. This pump system 270 includes a rigid container 271 defining a chamber 272 that may include a slider assembly 273 that separates the chamber 272 into a liquid portion 272A and an air portion 272B and to which springs may be attached to strengthen the rebound force of the deflectable diaphragm 275 covering the container 271. The container 271 includes a plurality of inlets and outlets leading into and out of the chamber of the container 272. There is a renal pelvis inlet 276 that is connectable to a catheter 277 leading to the renal pelvis. This inlet 276 includes a check valve 278 directed such that urine from the renal pelvis may flow into the container 271 but may not flow out of the container 271 through the renal pelvis inlet 276.

A second inlet is the therapeutic agent inlet 278 that is attachable to a source 279 of therapeutic agent. This inlet 278 also includes a check valve 280 directed such that therapeutic agent may flow into the container 271 from the source 279 but may not flow out of the container 271 through the therapeutic agent inlet 278.

One of the outlets from the container 271 is a renal pelvis outlet 281 that leads from the container 271 to the renal pelvis via a catheter. The outlet 281 includes a check valve 282 that prevents retrograde flow through the outlet back into the container 2271. Another outlet is an exhaust outlet 283. The exhaust outlet 283 allows air to escape from the air portion 272B of the chamber 272 when the deflectable diaphragm 275 is depressed.

Additionally, known pump designs could also be used such as piezo-electric disc pumps, mini diaphragm pumps, and the like.

Methods

Having described the various mechanical components of the invention and how they interact with each other, attention can be drawn now to how to use these components to improve kidney function.

In general, the excretion of fluid from the body is driven by filtration at the glomerulus as well as reabsorption and secretion in the peritubular capillaries. The dynamics by which this happens can be modeled using equations derived from Starling's Law. The Starling equation governs the formation of primary filtrate. The Starling equation can be broken into mechanical components and chemical components. The mechanical components deal with the hydrostatic pressure inside the of the Bowman's capsule and can be modified by a vacuum in the renal pelvis. A vacuum in the renal pelvis will reduce the pressure in Bowman's capsule, increasing the flux of primary filtrate and increasing glomerular filtration rate (GFR).

The chemical component of Starling's equation is also modified by the invention. By increasing the oncotic pressure in Bowman's capsule, the amount of primary filtrate is increased, augmenting GFR using known physics of filtration, as explained below. Further, the perfusion of agents may modify the increase in the chemical reflection coefficient in the Starling equation, thus limiting the amount of reabsorption and increasing the amount of filtrate and GFR.

Additionally, the perfusion of agents could impact the distal urine collecting areas of the nephron. ADH-antagonist perfusion is a targeted drug delivery method to reduce the amount of mediated water reabsorption that is typically upregulated in ADHF, CHF, CKD, and AKI. Other infusion could regulate the medullary gradient that is thrown out of sync in ADJF, CHF, CKD, and AKI. This allows the paradigm of “water follows sodium” to work to the patient's advantage, as sodium is being placed in the urine collecting area as opposed to the interstitium.

For example, as blood flows through the kidneys, various filtrate exchanges take place between the renal capillary structure and the Bowman's capsules of the nephrons. Using the following variables from Starling's law, various filtrate flow rates can be calculated:

| Term | Definition |
|---|---|
| J | Flow rate of filtrate |
| | [mL/min] |
| K_{f} | Filtration coefficient |
| | $\frac{[ \frac{mL}{min} ]}{mmHg}$ |
| P_{c} | Capillary hydrostatic |
| | pressure |
| | [mmHg] |
| Pᵢ | Bowman's space |
| | hydraulic pressure |
| | [mmHg] |
| σ | Reflection coefficient |
| | [Unitless; 0-1] |
| π_{c} | Oncotic pressure in |
| | capillary |
| | [mmHg] |
| π_{g} | Oncotic pressure in |
| | Bowman's Space, w/ |
| | glycocalx |
| | [mmHg] |

First, blood flows through the afferent arteriole and enters glomerular capillaries, which are contained in a Bowman's capsule of the kidney. Filtration of the blood occurs as the blood is flowing through the glomerular capillaries. The flow of filtrate from the glomerular capillaries into the Bowman's capsule can be modeled by a reduced, modified Starling Law as follows:

$$J \approx {K}_{f} \cdot ( {P}_{c} - {P}_{i} )$$

Next, as the filtrate travels from the Bowman's capsule to the renal tubule, reabsorption of some of the filtrate occurs between the renal tubule and the peritubular capillaries. The rate at which this occurs is modeled with a modified Starling law as follows:

$$J = {K}_{f} \cdot ( [ {P}_{c} - {P}_{i} ] - σ [ {}_{c} - {π}_{g} ] )$$

Finally, excretion happens from the peritubular capillaries back into the renal tubule according to the above modified Starling law. Thus, the total urinary excretion rate from the blood is equal to the filtration rate minus the reabsorption rate plus the secretion rate.

The total urinary excretion rate can be improved according to the invention by creating a vacuum in the renal pelvis and by infusing therapeutic agents. Creating a vacuum in the renal pelvis or ureter could result in a decrease in the Bowman's space hydraulic pressure, which is subtracted from the capillary hydrostatic pressure in the above equations. Thus, a decrease in the Bowman's space hydraulic pressure increases the overall urinary excretion rate.

Creating a vacuum in the ureter and/or renal pelvis may have a further effect that is understood by examining the formula for the filtration coefficient:

$${K}_{f} = {L}_{p} \cdot S {A}_{organ}$$

$${L}_{p} = \frac{N \cdot C \cdot {r}^{4}}{Δ X \cdot η}$$

In which:

| Term | Definition |
|---|---|
| K_{f} | Filtration coefficient |
| | $\frac{[ \frac{mL}{min} ]}{mmHg}$ |
| Lₚ | Hydraulic conductance |
| N | Number of pores/cm² |
| | [1/cm²] |
| C | Constant |
| r | Radius of filtering pores/slits |
| | [cm] |
| ΔX | Thickness of capillary wall |
| | [cm] |
| η | Fluid viscosity |
| | $[ \frac{N}{{m}^{2}} \cdot S ]$ |

A vacuum in the renal pelvis could increase the radius r of the filtering pores, or the width of the podocyte filtration slits. An increase in this variable is raised to the fourth power in the formula for the filtration coefficient.

The benefits of using therapeutic infusates also may also be demonstrated by further examination of the above equations. For example, a deeper understanding of the reflection coefficient component of the Starling Force Law can be used to further reduce absorption, resulting in greater total urinary excretion rate. The reflection coefficient is calculated using the following formula:

$$σ = 1 - \frac{{C}_{i}}{{C}_{c}}$$

In which:

| Term | Definition |
|---|---|
| K_{f} | Filtration coefficient |
| | $\frac{[ \frac{mL}{min} ]}{mmHg}$ |
| σ | Reflection coefficient |
| | [Unitless; 0-1] |
| Cᵢ | Concentration of particular protein |
| | in Bowman's capsule |
| C_{c} | Concentration of particular protein |
| | in capillary |

It is likely possible to decrease the reflection coefficient through infusion of polycations such as protamine, thereby decreasing absorptive flux. This has been demonstrated in isolated glomeruli. By infusing solutes (e.g. polycations or other highly oncotic pressure solutes), the concentration of solute in the filtrate space is increased, resulting in a decrease of the reflection coefficient and thereby increasing the total amount of filtrate.

Infusion of therapeutic agents, such as polycations and/or protamine, could also impact the filtration coefficient of the glycocalyx, increasing the total urinary excretion rate. The modified Starling law accounts for the presence of glycocalyx, as detailed in the below charts comparing showing the Starling principle with and without the presence of glycocalyx:

Furthermore, it is possible that modifying the temperature, pH, and tonicity of the infusate could have an impact that improves renal function. It can also be perceived that in situ modifications such as electrical pulses in the renal pelvis or pulsed magnetic polarizations in the renal pelvis could improve renal function. For example, a pulsed magnetic field was shown to improve cerebral blood flow and tissue oxygenation in the cerebral space in rats. Bragin, Denis, et al. Pulsed Electromagnetic Field (PEMF) Mitigates High Intracranial Pressure (ICP) Induced Microvascular Shunting (MVS) in Rats. Acta Neurochir Suppl. 126, 93-95 (2019). It is plausible, for example, that a pulsed electromagnetic field in situ in the renal pelvis, urine collecting system, or general kidney increases renal blood flow and improves kidney oxygenation, resulting in improved kidney performance. Another embodiment could employ an electrode placed within the venous system and another electrode placed within the urinary system (renal pelvis, ureter, or bladder) to create an electrical potential. This potential could drive ion flow into the renal pelvis.

Turning to FIG. 18 for reference, one method of the present invention involves placing an introducer sheath 301 into the bladder through the urethra of a patient. The introducer sheath 301 may be used then to route guidewires 302 and 304 through the ureters into the renal pelvis cavities of each kidney. Next, catheters 306 and 308 are advanced over the guidewires until the distal ends 310 and 312 of the catheters 306 and 308 are located in the renal pelvises.

Next, balloons 314 and 316 are inflated to occlude the ureters such that a vacuum may be drawn in the renal pelvises without drawing urine back into the kidneys via the ureters. Once inflated, the catheters 306 may be proximally attached to a suction source, such as a pump, to remove fluid from the kidneys and to establish a negative pressure in the renal pelvises. Creating a vacuum in the ureteral pelvises increases renal blood flow. This method may be performed contralaterally or ipsilaterally.

FIG. 19 illustrates another embodiment of a method of the invention. This method involves placing an introducer sheath 320 into the bladder through the urethra of a patient. The introducer sheath 320 may be used then to route guidewires 322 and 324 through the ureters into the renal pelvis cavities of each kidney. Next, catheters 326 and 328 are advanced over the guidewires until the distal ends 330 and 332 of the catheters 326 and 328 are located in the renal pelvises.

Next, balloons 334 and 336 are inflated to occlude the ureters such that the renal pelvises are isolated from the ureters. Once inflated, a therapeutic solution is injected into the renal pelvises at various rates for a various time. The rates and times could be adjusted to one or more physiologic parameters. Once injected, a settling time is provided to allow the hypertonic to infiltrate the collecting ducts that lead to the renal pelvises. Finally, a vacuum is drawn through the catheters to aspirate and offload urine to the bladder.

This method incorporates benefits determined in various studies. For example, renal blood flow has been shown to increase by 25 percent immediately after transient bilateral ureteral obstruction (for up to 1-2 hours) due to hypothesized efferent vasodilation. Loo, M. H., Felsen, D., Weisman, S., Marion, D. N. & Vaughan, E. D. Pathophysiology of obstructive nephropathy. Kidney Int. 18, 281-292 (1980). GFR is only 80% of normal after transient bilateral ureteral obstruction (for up to 1-2 hours), suggesting limited filtration impact. (Id.) Additionally, fluid resorption decreased in obstructed kidneys. Hanley, M. J. Studies on acute disease models. Kidney Int. 22, 536-545 (1982). Relief of bilateral ureteral obstruction caused marked increase in sodium and water excretion. (Loo, et al.) The main inference is that obstruction increases ureteral pressure, which may reduce GFR and force solutes into the interstitial space of the kidney, causing a vicious cycle of reduced filtration and increased reabsorption. This invention infers that causing the opposite condition, namely inducing a negative pressure and perfusing the urine collecting system with polyurea-inducing and other general agents that promote solute clearance could substantially improve kidney function.

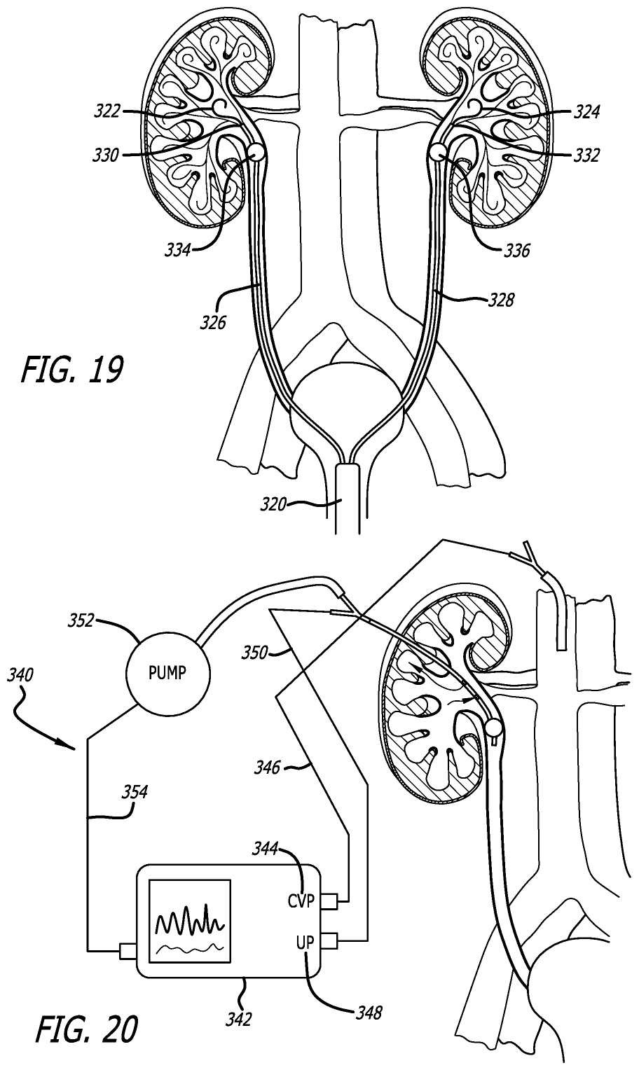

One aspect of the use of the non-manual pump systems described herein is maintaining a desired pressure in the kidneys. A method for doing so is shown in FIG. 20. Control is established by first applying therapy to the renal pelvis via negative pressure or via a therapeutic agent such as a diuretic, an ADH antagonist, dialysate, or salt injection. Next, various physiologic signals are monitored, such as central venous pressure, urine output, serum creatinine, creatinine clearance, urea, pressure in the renal pelvis, etc. Finally, the therapy level is adjusted to reach the target physiologic output.

FIG. 20 shows an example of a feedback loop 340 that can be used to practice this method. In this example, pressure is being used as an input for the feedback loop 340. Pressure readings are taken using a pressure sensor 342. Inputs into the sensor 342 include central venous pressure CVP 344 provided via a central venous catheter 346, and ureter pressure UP 348, which is sampled from the therapy catheter 350. The pump 352 receives an output 354 from the sensor 342 such that it can adjust speed to achieve a desired pressure.

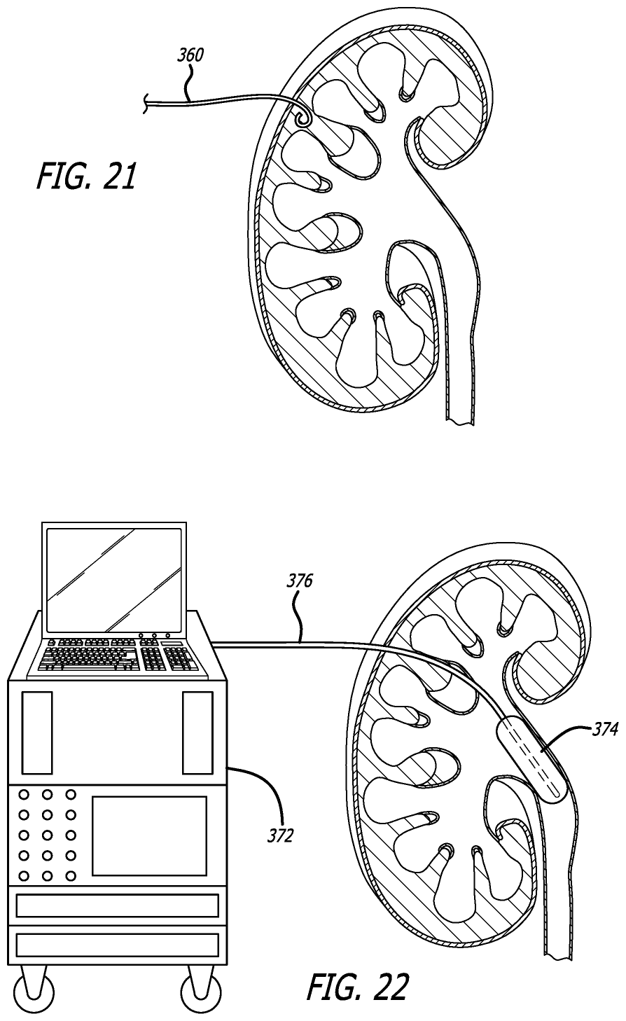

FIG. 21 illustrates a method in which intervention is achieved inside the renal tissue, rather than in the renal pelvis. This method involves using a suction catheter 360 to create a low-pressure zone inside the renal tissue. Additionally, an ADH-antagonist, a diuretic, or a dialysate could be injected through the catheter 360 inside the tissue.

FIG. 22 illustrates a method in which the system of FIG. 8 is used with an implantable pump 220, such as that shown in FIG. 13 or an external unit 372 such as that shown in FIG. 22. The pump is used to slowly inflate and rapidly deflate the balloon 374 such that the flow of fluid through the ureter is assisted or enhanced. The catheter 376 is positioned such that the balloon 374 is located within the ureter. When the balloon 374 is inflated, pressure does not build on the kidney or upstream side of the balloon 374 due to the slow inflation of the kidney. When the balloon is rapidly deflated, the pressure is quickly released and the flow through the ureter is greater than a steady state flow that would occur if the balloon were not inflated. It is also possible that the balloon 374 is rapidly inflated and rapidly deflated. The rapid inflation would cause an increase in ureteral pressure; rapid reversal of this increase through a rapid deflation of the balloon could cause a sharp relief of the ureteral obstruction, causing significant post-obstructive diuresis. As it is shown in FIG. 22, the catheter is routed through the renal capsule, past the renal pelvis, and into the ureter. The catheter could also be perceivably be routed through the urethra into one or both ureters or renal pelvises.

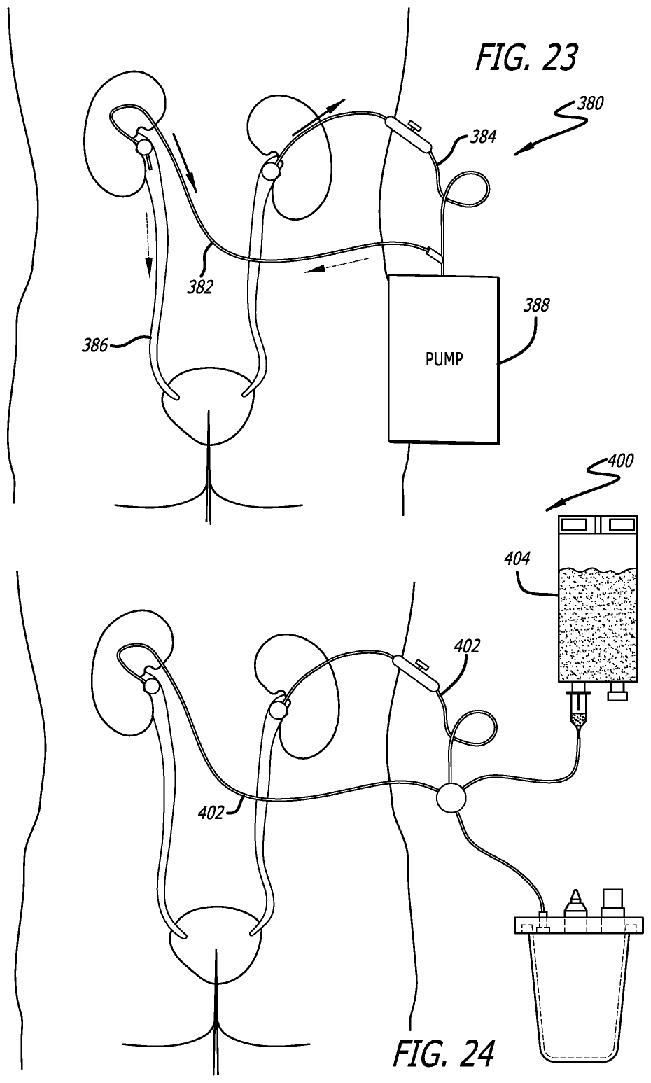

FIG. 23 depicts a method 380 that uses two suction flow paths 382 and 384, as indicated by the arrows, and one drainage path 386 for urine to flow to the bladder. In cases where both kidneys need to be intervened, a double nephrostomy can be performed. In one kidney, a standard nephrostomy tube can be placed. In the other kidney (the left kidney in FIG. 23), a ureter is catheterized and there is a lumen to suck out urine from the renal pelvis and another lumen distally that pushes the urine towards the bladder. The pump 388 would therefore vacuum out urine from both renal pelvises but then only reintroduce the urine back into one of the catheters and direct the urine towards the bladder.

FIG. 24 depicts a dual nephrostomy method 400. This method uses two catheters 402 extending between the kidneys and a nephrostomy bag 404. The catheters 402 could be connected to a pump that alternates between vacuum and infusion pressures. One option for the dual nephrostomy approach is that it could double the effectiveness of the therapy by alternating which kidney is exposed to vacuum and which is exposed to infusion, effectively ensuring that one kidney is always being exposed to vacuum. Another option for this approach is to help to mitigate a theoretical reno-reno reflex. In some conditions, this reflex seeks to maintain an average kidney function between the two kidneys (i.e. if one is underperforming the other will increase function to maintain the same total average function).

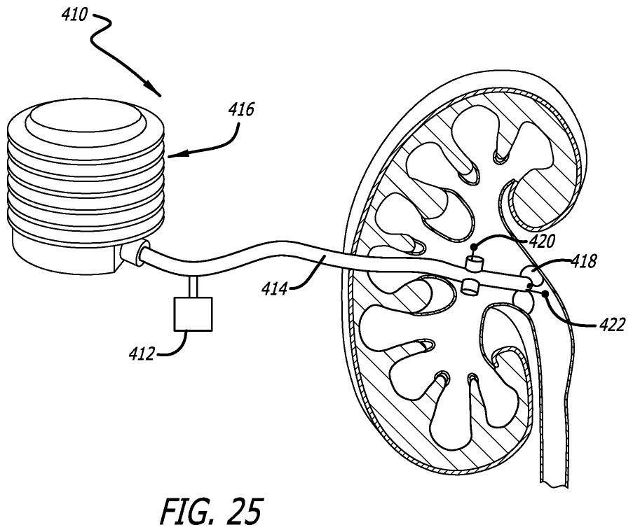

FIG. 25 depicts a method for determining adequate vacuum pressure. The method 410 uses a flow meter 412 tapped into a catheter 414 between the kidney and a suction device 416. The catheter 414 includes a distal occlusion balloon 418, a proximal pressure sensor 420 on one side of the balloon, and a distal pressure sensor 422 on the other side of the balloon. The method involves first reading the pressure measurements from the pressure sensors 420 and 422. Next, the balloon 418 is inflated and the pressure is again measured on the proximal side of the balloon 418 using the proximal pressure sensor 420 and on the distal side of the balloon 422. The urine output can be calculate using the measured pressure differential and by utilizing the known resistance of the tube to flow. Next, a determination is made whether the calculated urine output is adequate. The suction device is then adjusted accordingly depending on the urine flow. The balloon 418 is then deflated and the suction is again adjusted based on the flow. Flow may be enhanced by reducing the duration of the balloon inflation.

In some situations, it may be preferable for the referral physician to access the renal pelvis transvenously. The advantages of doing so include the leverage of existing access in ICU patients, and the ability to use a multi-lumen catheter to concurrently perform veno-venous dialysis, if needed. This leverages existing provider care pathways for AKI and integrates into standard practice.

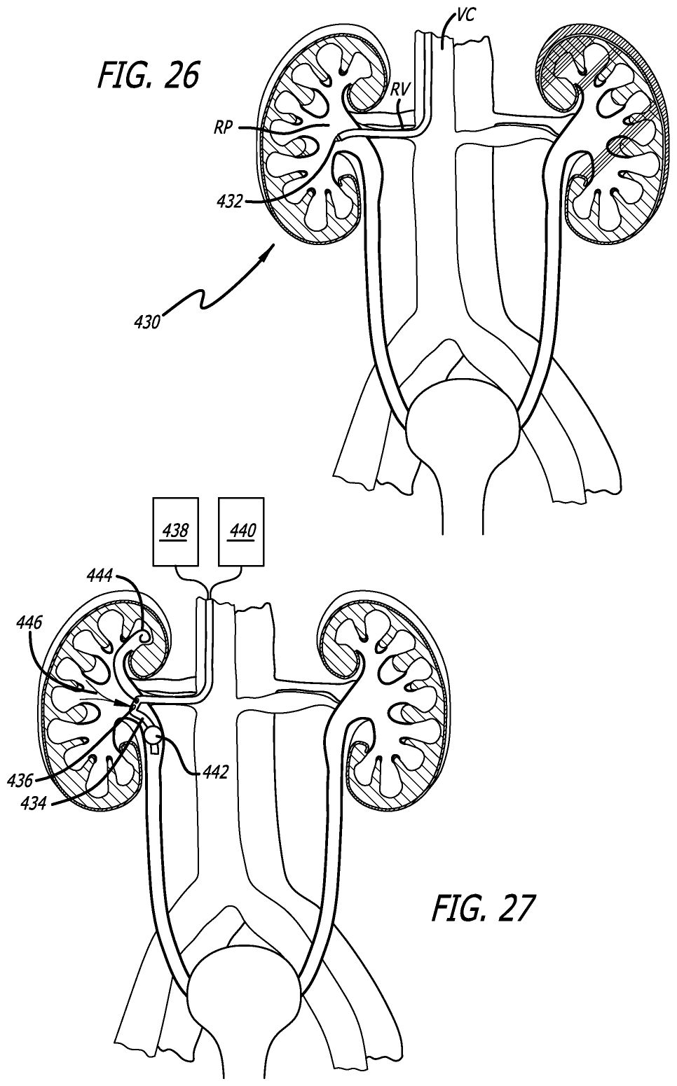

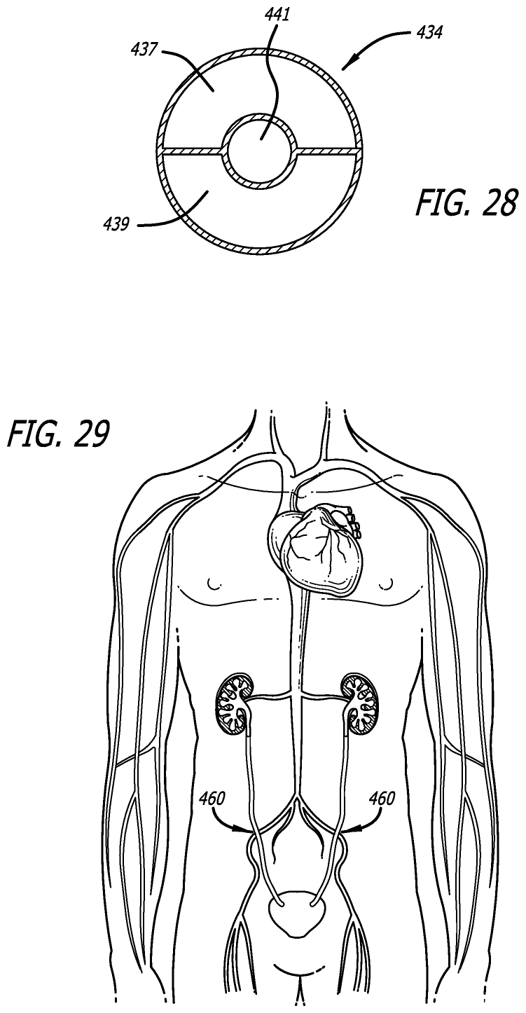

FIGS. 26-28 depicts a method 430 utilizing a transvenous approach to access the kidney. FIG. 26 shows the steps of accessing the renal pelvis. First, a needle 432 is navigated through the renal vein RV via the vena cava VC and into the renal pelvis RP. Next, as shown in FIG. 27, a therapy catheter 434 is advanced over the needle 432 and the needle 432 is retracted. The therapy catheter 434 may be one of the catheters described herein and may include perfusion/vacuum holes 436. A proximal end of the therapy catheter 434 is connected to a hemodialysis machine 438 and a renal pelvis decompression device 440. A cross section of an embodiment of a catheter 434 is shown in FIG. 28. The catheter 434 in this embodiment may include a venous blood in lumen 437, a venous blood out lumen 438 and a renal pelvis vacuum and/or perfusion lumen 440. This central lumen 440 could serve as both a vacuum and a perfusion lumen or could be bifurcated such that one side becomes a vacuum lumen and the other side becomes a perfusion lumen.

After the needle 432 is retracted, a sealing element, such as a balloon 442 for example, is inflated, isolating the renal pelvis from the ureter. A combination therapy is then administered involving infusion of a therapeutic agent 444 and vacuum suction 446, both delivered via the catheter 434.

In addition to superior vena cava (central line-style) access, it is possible to access the renal pelvis via the femoral vein and create a puncture into the ureter to access the urine collecting system. In some cases this route may be preferred for navigating a chronic-indwelling renal pelvis decompression device. FIG. 29 is an anatomical diagram showing an optimal access point 460 where the femoral vein and the ureter are in close natural proximity.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method of improving kidney function in a patient comprising:
reducing pressure in a renal system of a least one kidney of a patient;
infusing a therapeutic agent into the renal system of the at least one kidney;
said reducing pressure and said infusing a therapeutic agent being conducted in combination during a therapeutic intervention on said patient.

2. The method of claim 1 wherein said reducing pressure and said infusing a therapeutic agent being conducted in combination comprises alternating between said reducing pressure and said infusing a therapeutic agent.

3. The method of claim 1 wherein reducing a pressure in a renal system of at least one kidney of a patient comprises occluding a ureter and drawing a vacuum in a renal pelvis.

4. The method of claim 1 wherein infusing a therapeutic agent into the renal system of the at least one kidney comprises inserting an infusion catheter through a renal capsule of the at least one kidney.

5. The method of claim 1 wherein reducing a pressure in a renal system of at least one kidney of a patient comprises inserting a vacuum catheter through a renal capsule of the at least one kidney.

6. The method of claim 5 wherein said vacuum catheter comprises a proximal end connected to a vacuum pump.

7. The method of claim 6 wherein said vacuum pump further serves as an infusion pump.

8. The method of claim 6 wherein said infusing a therapeutic agent comprises injecting a syringe containing therapeutic agent into an injection port on said catheter.

9. The method of claim 1 wherein reducing the pressure in the renal system of the at least one kidney of the patient comprises reducing the pressure in the renal system of both kidneys of the patient.

10. The method of claim 9 wherein infusing the therapeutic agent into the renal system of the at least one kidney comprises infusing the therapeutic agent into the renal system of both kidneys of the patient.

11. The method of claim 10 further comprising draining fluid vacuumed from the renal system of the at least one kidney into a bladder of the patient.

12. The method of claim 1 wherein at least one of reducing the pressure in the renal system and infusing a therapeutic agent comprises accessing the renal system with a catheter using a trans-vascular approach.

13-16. (canceled)

14. The method of claim 13 further comprising infusing an agent using said access route to further increase kidney function as a result of said agent.

15. The method of claim 14 wherein drawing the vacuum and infusing the agent are performed alternatingly.

16. The method of claim 13 wherein drawing the vacuum comprises:
advancing an occlusion balloon through the renal pelvis inflating the balloon such that a ureter is blocked; and,
pumping fluid out of the renal pelvis.

17. A method of improving kidney function of a patient comprising:
creating access to a renal pelvis region of a kidney of said patient through a wall of said kidney;
introducing a therapeutic agent into said pelvis region so as to increase kidney function as a result of said introduction of said therapeutic agent.

18. The method of claim 17 further comprising drawing a vacuum in said renal pelvis.

19. The method of claim 18 wherein introducing the therapeutic agent and drawing the vacuum occur simultaneously.

20. The method of claim 18 wherein introducing the therapeutic agent and drawing the vacuum occur alternatingly.

21. A system for improving kidney function of a patient comprising:
an access device configured for placement through a wall of a kidney to a renal pelvis region of said kidney;
a vacuum inducing component associated with said access device configured for drawing a vacuum in said renal pelvis region;
an infusion component associated with said access device configured for introducing a therapeutic agent into said renal pelvis region;
said access device, said vacuum inducing component and said infusion inducing component in combination, during operation, increasing kidney function.

22. The system of claim 21 further comprising an occlusion component configured to isolate a pressure in a ureter from a pressure in the renal pelvis region.

23. The system of claim 21 wherein said vacuum inducing component comprises a pump.

24. The system of claim 23 wherein said pump comprises a manual pump.

25. The system of claim 23 wherein said pump comprises an implantable pump.

26. The system of claim 21 wherein said access device comprises a catheter.

27. The system of claim 26 wherein said access device further comprises a guide wire.

28. The system of claim 27 wherein said access device further comprises a needle.

29. The system of claim 26 wherein said catheter comprises a multi-lumen catheter and wherein at least one of said lumens is associated with said vacuum inducing component and wherein at least one of said lumens is associated with said infusion component.

30. The system of claim 26 wherein said catheter comprises a balloon catheter having a lumen that terminates in holes proximal of a balloon and having a second lumen that passes through said balloon.

41. A system for improving kidney function of a patient comprising:
an access device configured for placement through a wall of a kidney to a renal pelvis region of said kidney;
an infusion component associated with said access device configured for introducing a therapeutic agent into said renal pelvis region;
said access device and said infusion inducing component in combination, in operation, increasing kidney function.

42. The system of claim 41 further comprising an occlusion component configured to isolate a pressure in a ureter from a pressure in the renal pelvis region.

43. The system of claim 41 wherein said vacuum inducing component comprises a pump.

44. The system of claim 43 wherein said pump comprises a manual pump.

45. The system of claim 43 wherein said pump comprises an implantable pump.

46. The system of claim 41 wherein said access device comprises a catheter.

47. The system of claim 46 wherein said access device further comprises a guide wire.

48. The system of claim 47 wherein said access device further comprises a needle.

49. The system of claim 46 wherein said catheter comprises a multi-lumen catheter and wherein at least one of said lumens is associated with said vacuum inducing component and wherein at least one of said lumens is associated with said infusion component.

50. The system of claim 46 wherein said catheter comprises a balloon catheter having a lumen that terminates in holes proximal of a balloon and having a second lumen that passes through said balloon.

51-65. (canceled)

66. A system for improving kidney function of a patient comprising:
a percutaneous access device configured for placement in a renal pelvis region of said kidney;
an infusion component associated with said access device configured for introducing a therapeutic agent into said renal pelvis region;
said access device and said infusion inducing component in combination, in operation, increasing kidney function.

67. The system of claim 66 further comprising an occlusion component configured to isolate a pressure in a ureter from a pressure in the renal pelvis region.

68. The system of claim 66 wherein said vacuum inducing component comprises a pump.

69. The system of claim 68 wherein said pump comprises a manual pump.

70. The system of claim 68 wherein said pump comprises an implantable pump.

71. The system of claim 66 wherein said access device comprises a catheter.

72. The system of claim 71 wherein said access device further comprises a guide wire.

73. The system of claim 72 wherein said access device further comprises a needle.

74. The system of claim 71 wherein said catheter comprises a multi-lumen catheter and wherein at least one of said lumens is associated with said vacuum inducing component and wherein at least one of said lumens is associated with said infusion component.

75. The system of claim 71 wherein said catheter comprises a balloon catheter having a lumen that terminates in holes proximal of a balloon and having a second lumen that passes through said balloon.

76. A method for improving kidney function comprising:
creating a vacuum within a renal pelvis of a kidney of a patient, thereby drawing urine out of renal tissue surrounding the renal pelvis;
infusing a therapeutic agent into the renal pelvis to supplement the effects of the vacuum.

77. The method of claim 76 wherein creating the vacuum within the renal pelvis comprises introducing a suction catheter percutaneously into the renal pelvis.

78. The method of claim 77 further comprising occluding the ureter prior to creating the vacuum.

79. The method of claim 76 further comprising directing urine from the renal pelvis to the bladder.

80. The method of claim 76 wherein creating a vacuum within the renal pelvis comprises:
placing a one-way valve within a ureter that prevents retrograde flow from a bladder toward the kidney;
inflating a balloon within the renal pelvis, thereby reducing a volume within the renal pelvis thus displacing urine contained therein through the valve and toward the bladder; and,
deflating the balloon, thereby increasing a volume in the renal pelvis and thus creating a temporary vacuum within the renal pelvis until the urine drawn out of the renal tissue surrounding the renal pelvis refills the renal pelvis.

81. The method of claim 76 wherein creating the vacuum within the renal pelvis comprises:
occluding a ureter associated with the kidney; and,
using a pump to remove fluid from the renal pelvis.

82. The method of claim 81 further comprising directing the fluid removed from the renal pelvis to a bladder through the ureter.

83. (canceled)

84. The method of claim 76 further comprising using electrical pulses in the renal pelvis to improve renal function.

85. The method of claim 76 further comprising using pulsed magnetic polarizations in the renal pelvis to improve renal function.

86. A system for improving kidney function of a patient comprising:
a percutaneous catheter having a proximal end and a distal end;
a pump connected to one of the ends; and,
a fluid control mechanism that directs fluid manipulated by the pump out of a renal pelvis of the kidney such that a vacuum is established in the renal pelvis resulting in increased fluid production by the kidney.
